(19) United States
(12) Patent Application Publication
(10) Pub. No.: US 2009/0264985 A1
(43) Pub. Date: Oct. 22, 2009

(54) Branch Vessel Suture Stent System and Method

(75) Inventor: Walter Bruszewski, Guerneville, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/104,641

(22) Filed: Apr. 17, 2008

(51) Int. Cl. A61F 2/06

(52) U.S. Cl. 623/1.18; 623/1.51; 623/1.35; 623/1.36

(57) ABSTRACT

A branch vessel suture stent system and method including a branch vessel suture stent having a stent body having a first end, a second end, and a central axis, the first end having a first periphery; and shape memory hooks disposed about the first periphery, each of the shape memory hooks being attached to the first periphery at an attachment point, the shape memory hooks being elongated in a stressed state and looped in a parent state, each of the shape memory hooks defining a loop plane in the parent state. The shape memory hooks are substantially parallel to the central axis in the stressed state, and the first periphery at the attachment point for each of the shape memory hooks is substantially orthogonal to the loop plane for each of the shape memory hooks in the parent state. The delivery system allows the main body of the branch device to expand while maintaining the hooks in an undeployed configuration using individual hypotubes. Then once the body of the suture stent has expanded allowing the suture stent shape memory hooks to be released and engage the main graft body and/or surrounding tissue.

### TECHNICAL FIELD

The technical field of this disclosure is medical implantation devices, particularly, a branch vessel suture stent system and method.

### BACKGROUND OF THE INVENTION

Wide ranges of medical treatments have been developed using endoluminal prostheses, which are medical devices adapted for temporary or permanent implantation within a body lumen, such as naturally occurring or artificially made lumens. Examples of lumens in which endoluminal prostheses may be implanted include arteries such as those located within coronary, mesentery, peripheral, or cerebral vasculature; veins; gastrointestinal tract; biliary tract; urethra; trachea; hepatic shunts; and fallopian tubes. Various types of endoluminal prostheses have also been developed with particular structure to modify the mechanics of the targeted luminal wall.

A number of vascular devices have been developed for replacing, supplementing, or excluding portions of blood vessels. These vascular devices include endoluminal vascular prostheses and stent grafts. Aneurysm exclusion devices, such as abdominal aortic aneurysm (AAA) devices, are used to exclude vascular aneurysms and provide a prosthetic lumen for the flow of blood. Vascular aneurysms are the result of abnormal dilation of a blood vessel, usually from disease or a genetic predisposition, which can weaken the arterial wall and allow it to expand. Aneurysms can occur in any blood vessel, but most occur in the aorta and peripheral arteries, with the majority of aneurysms occurring in the abdominal aorta. An abdominal aortic aneurysm typically begins below the renal arteries and extends into one or both of the iliac arteries.

Aneurysms, especially abdominal aortic aneurysms, have in the past been treated using open surgical procedures in which the diseased vessel segment is bypassed and repaired using a sewn in artificial vascular graft. While open surgery is an effective surgical technique in light of the risk of a fatal abdominal aortic aneurysm rupture, the open surgical technique suffers from a number of disadvantages. The surgical procedure is complex, requires a long hospital stay, requires a long recovery time, and has a high morbidity and mortality rates. Less invasive devices and techniques have been developed to avoid these disadvantages. Tubular endoluminal prostheses that provide a conduit or conduits for blood flow while excluding blood flow to the aneurysm site are introduced into the blood vessel using a catheter in a less or minimally invasive technique. The tubular endoluminal prosthesis is introduced in a small diameter crimped condition and expanded at the aneurysm. Although often referred to as stent grafts, these tubular endoluminal prostheses differ from covered stents in that they are not used to mechanically prop open natural blood vessels. Rather, they are used to secure an artificial conduit in a sealing engagement with the vessel wall without further opening the abnormally dilated natural blood vessel.

Stent grafts for use in abdominal aortic aneurysms typically include a support structure supporting woven or interlocked graft material. Examples of woven graft materials are woven polymer materials, e.g., Dacron, or polytetrafluoroethylene (PTFE). Interlocked graft materials include knit, stretch, and velour materials. The graft material is secured to the inner or outer diameter of the support structure, which supports the graft material and/or when functioning as a seal holds it in place against a surrounding vessel wall. In this configuration the stent graft is secured to a vessel wall above and below the aneurysm. A proximal spring stent of the stent graft can be located above the aneurysm to provide a radial force which engages the vessel wall and provides an outward force to seal the proximal end of the stent graft to the vessel wall. The proximal spring stent can include anchor pins to puncture the vessel wall and further secure the stent graft in place.

One impediment in using stent grafts high in the abdominal aorta is the need to maintain blood flow to the renal arteries and superior mesenteric artery when the only region suitable for sealing the proximal end of the stent graft to the wall of the aorta is superior to these visceral arteries. An estimated ten percent of AAA cases amenable to endovascular repair require suprarenal fixation, potentially cutting off blood to the kidneys and intestine. One proposed solution to this problem has been to provide branched conduits from the stent graft whose main body covers these branches to perfuse the renal arteries and superior mesenteric artery.

In such arrangements, a joint created in situ between the stent graft and the branched conduit is prone to leakage, reducing flow to the branch vessels and continuing to pressurize the aneurysmal sac. One approach to this problem has been to custom make a stent graft with branched conduits for a particular patient, so that the branch conduit seal is fabricated before the stent graft is deployed in the patient. This approach has its own problems, however, since each stent graft is different, requiring a customized stent graft constructed using individualized personal measurements and construction. In addition, the bulky customized stent grafts are difficult to deploy, since the branch conduits are attached before deployment and thus make the device so much more bulky. The efficacy of such branched customized stent grafts is yet to be proven.

Another approach to the problem of branch conduit joint leakage has been to fenestrate the graft material in situ after the stent has been deployed, deploy a covered branch vessel stent in the fenestration to provide a flow path (conduit) between the main stent graft lumen and the visceral artery, and form the seal in place. This approach has problems because complicated sealing mechanisms are required and limited working space is available in the vessel. One approach has been to flare the end of the branch vessel stent. Flaring is however technically complex and time consuming to implement. Another approach has been to use a grommet or fitting crimped across the graft material, but such fittings are complex and difficult to deploy.

Another problem is fixation of the branch vessel stent at the visceral artery. If the stent graft shifts after being implanted, the branch vessel stent may no longer be aligned with the visceral artery and the length of the branch vessel stent in the visceral artery may be reduced. The patency of the branch vessel stent may be reduced if twisting of the branch vessel stent causes it to partially collapse.

It would be desirable to have a branch vessel suture stent system and method that would overcome the above disadvantages.

### SUMMARY OF THE INVENTION

One aspect according to the present invention provides a branch vessel suture stent having a stent body having a first end, a second end, and a central axis, the first end having a first periphery; and shape memory hooks disposed about the first periphery, each of the shape memory hooks being attached to the first periphery at an attachment point, the shape memory hooks being elongated in a stressed state and looped in a parent state, each of the shape memory hooks defining a loop plane in the parent state. The shape memory hooks are substantially parallel to the central axis in the stressed state, and the first periphery at the attachment point for each of the shape memory hooks is substantially orthogonal to the loop plane for each of the shape memory hooks in the parent state.

Another aspect according to the present invention provides a branch vessel suture stent system including a suture stent delivery catheter; and a branch vessel suture stent operably attached to the suture stent delivery catheter, the branch vessel suture stent having a stent body having a first end, a second end, and a central axis, the first end having a first periphery; and shape memory hooks disposed about the first periphery, each of the shape memory hooks being attached to the first periphery at an attachment point, the shape memory hooks being elongated in a stressed state and looped in a parent state, each of the shape memory hooks defining a loop plane in the parent state. The shape memory hooks are substantially parallel to the central axis in the stressed state, and the first periphery at the attachment point for each of the shape memory hooks is substantially orthogonal to the loop plane for each of the shape memory hooks in the parent state.

Another aspect according to the present invention provides a method of stenting a branch vessel off a main vessel, the method including providing a main vessel stent graft having main vessel stent graft material and a main vessel stent graft lumen; deploying the main vessel stent graft in the main vessel; providing a branch vessel suture stent in a stressed state, the branch vessel suture stent having a stent body and shape memory hooks, the stent body having a first end including a first periphery, the shape memory hooks being disposed about the first periphery; advancing the branch vessel suture stent through a fenestration in the main vessel stent graft to locate the stent body in the branch vessel and the shape memory hooks in the main vessel stent graft lumen; and allowing the seal portion of the stent to expand and then relaxing each of the shape memory hooks to engage the main vessel stent graft material and form a loop.

Another aspect according to the present invention provides a branch vessel suture stent for use in a branch vessel with a main vessel stent graft having main vessel stent graft material, the branch vessel suture stent including means for stenting the branch vessel, the stenting means having a central axis; and means for hooking the main vessel stent graft material, the hooking means being connected to the stenting means and having a stressed state and a parent state. Each of the hooking means is substantially parallel to the central axis in a compressed state and then as the stent reaches its initial deployment configuration in the stressed state the hooking means are released to form a loop in the parent state.

The foregoing and other features and advantages according to the invention will become further apparent from the following detailed description read in conjunction with the accompanying drawings. The detailed description and drawings are merely illustrative, rather than limiting in scope.

### BRIEF DESCRIPTION OF THE DRAWINGS

FIGS. 1A-1B are a side and close up cross sectional view, respectively, of a branch vessel suture stent system;

FIGS. 2A-2D are side and end views of a branch vessel suture stent;

FIG. 3 is a close up view of a shape memory hook for a branch vessel suture stent;

FIGS. 4A-4D are schematic diagrams of vessel/graft engagement of a branch vessel suture stent in accordance with the present invention;

FIG. 5 is a schematic diagram of vessel/graft engagement including suture stent graft material of a branch vessel suture stent;

FIG. 6 is a schematic diagram of graft engagement of a branch vessel suture stent;

FIGS. 7A-C are schematic views of the steps of deployment of a branch vessel suture stent;

FIGS. 8A-B are alternate views showing the deployment of a branch vessel suture stent;

FIGS. 9 is a side view of a branch vessel suture stent with suture stent graft material in accordance with the present invention; and

FIG. 10 is a flowchart of a method of stenting a branch vessel with a branch vessel suture stent.

### DETAILED DESCRIPTION

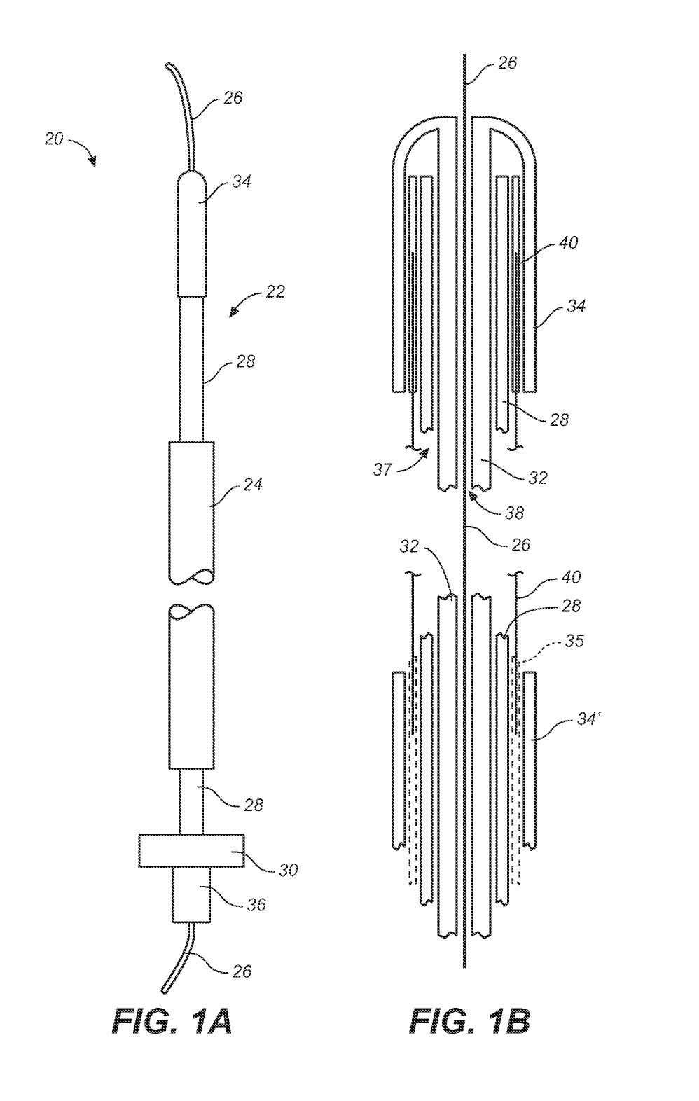

FIGS. 1A-1B are side and close up cross sectional views, respectively, of a branch vessel suture stent system. The branch vessel suture stent system 20 includes a suture stent delivery catheter 22 for delivering and deploying a branch vessel suture stent 40, a steerable catheter 24, and a guidewire 26. The suture stent delivery catheter 22 includes an outer shaft 28, an inner shaft 32, and a sheath (distal sheath 34 and proximal sheath 34′). The distal sheath 34 restrains the branch vessel suture stent 40 in a compressed (stressed) state until the branch vessel suture stent 40 is in position for deployment. When the proximal sheath 34′ is moved proximally to expose the hypotubes containing the hooks, the positioning of the proximal end of the suture stent 40 can be verified. Then the distal sheath 34 is moved distally to release the body of the branch vessel suture stent 40 to allow it to relax toward its parent state. The suture stent is prevented from moving forward with the sheath by engagement elements fixed to the inner shaft such as those shown in: US 20060184227A1, US2004/204749, and U.S. Pat. No. 6,607,551, all of which are incorporated herein by reference in their entirety. When the distal sheath 34 is moved distally to release the body of the suture stent, the proximal sheath 34′ is held stationary, which as the distal sheath 34 is retracted causes the proximal end of the suture stent, particularly its hooks to expand radially, while still held constrained (relatively straight and not looped) by the hypotube 35 bundle. Each individual hypotube in the circumferential bundle expands radially as the proximal sheath 34′ is optionally further retracted. Once the radial position of the proximal end of the suture stent is assured, the hypotubes are retracted to release the hooks.

The outer shaft 28 can be operably connected to a catheter handle 30 for advancing the branch vessel suture stent to the deployment site. In one embodiment, the outer shaft 28 has an outer shaft lumen 37 for receiving the inner shaft 32. The inner shaft 32 can be operably connected to a sheath handle 36 to move the sheath 34 axially and release the distal portion of branch vessel suture stent 40. In one embodiment, the inner shaft 32 has a guidewire lumen 38 for receiving the guidewire 26. The guidewire 26 can be advanced to the deployment site, such as a branch vessel, and the suture stent delivery catheter 22 advanced to the deployment site over the guidewire 26. The proximal portion of the suture stent 40, i.e., its hook portions are sleeved with separate hypotubes 35 (shown with dashed lines) when a handle element (not shown) attached to the hypotubes is retracted, the hook elements are released.

The branch vessel suture stent system 20 can be used to deploy a branch vessel suture stent at the ostium of a branch vessel connected to a main vessel. Exemplary deployment of a branch vessel suture stent in an abdominal aortic aneurysm proceeds as follows.

The main vessel stent graft is deployed in the main vessel. The steerable catheter is advanced to the abdominal aortic aneurysm through the femoral artery, the carotid artery, or the subclavian artery. The catheter is guided to the location of the aneurysm with X-ray or fluoroscopic data and a main vessel stent graft is advanced to the aneurysm. A guidewire is prepositioned in the vessel to guide the catheter to aid in deployment of the main vessel stent graft. A main vessel stent graft is initially deployed in and spanning the aneurysm. The distal end of the suture stent delivery catheter 22 is advanced to the branch vessel, such as a renal artery, over a pre-positioned guidewire 26. The branch vessel can be approached through a fenestration in the main vessel stent graft material of the main vessel stent graft. In one embodiment, the fenestration is made in the main vessel stent graft before the main vessel stent graft is inserted in the patient. In another embodiment, the fenestration is made in situ after the main vessel stent graft is deployed in the main vessel. The branch vessel suture stent 40 has a stent body and a number of shape memory hooks at the proximal end of the stent body. The sheath 34 is positioned through the fenestration so that the stent body is within the branch vessel and the shape memory hooks are still within the main vessel stent graft lumen. The sheath 34 is moved distally so that as the branch vessel suture stent 40 is released from the constraint of the sheath it (expands) changes from the stressed state to the parent state. The stent body expands in diameter and the shape memory hooks if unrestrained would loop toward the branch vessel to engage the main vessel stent graft material of the main vessel stent graft or the main vessel stent graft material of the main vessel stent graft and the ostium of the branch vessel. In the parent state, the shape memory hooks form loops. However, since the branch vessel suture stent 40 is transported to the delivery site in a compressed (small delivery catheter) diameter, looping of the memory hooks at the small diameter would cause the tips of the hooks to engage at a related small diameter. So in this instance a bundle (array) of hypotubes (one hypotube 35 for each hook) spaced around a tubular perimeter, each hypotube 35 individually having been slipped over (as a sleeve) or surrounding a respective corresponding hook loop to restrain (prevent) the hook loop from turning into a loop, while still in small body diameter. As the sheath 34 is moved off the stent 40 to allow it to fully radially expand and the main body portion of the stent establishes a deployed position as it expands and contacts the surrounding vessel and branch opening of the main stent graft body. Once the sheath has been removed and the body of the stent 40 has fully expanded the ends of the hypotubes still holding the hooks substantially straight, also expand to the full diameter of the branch vessel. Then the outer shaft to which the radially expanded hypotubes are articulably connected is retracted to release the hook loops to from their restraint and allow the loops to engage and secure themselves in the surrounding tissues and graft material. Once the branch vessel suture stent 40 is expanded fully and released to its parent state, the sheath 34 can be pulled back through the lumen of the branch vessel suture stent 40, and the suture stent delivery catheter 22, the guidewire 26, and the steerable catheter 24 removed from the patient.

Those skilled in the art will appreciate that the approach for deployment of the branch vessel suture stent can be made via externally accessible branch vessel, such as carotid, to the main vessel for particular applications, rather than the approach from the main vessel toward the branch vessel described above. For example, when a main vessel stent graft is deployed in the aortic arch as the main vessel, the suture stent delivery catheter can deliver the branch vessel suture stent through the right common carotid artery, the left common carotid artery, or the left subclavian artery as the branch vessels. When the approach is from the branch vessel, the shape memory hooks and hypotube mechanism holding them are disposed toward the distal end of the suture stent delivery catheter and the sheath is retracted toward the proximal end to release the branch vessel suture stent, while the hypotube mechanism is moved forward to be released.

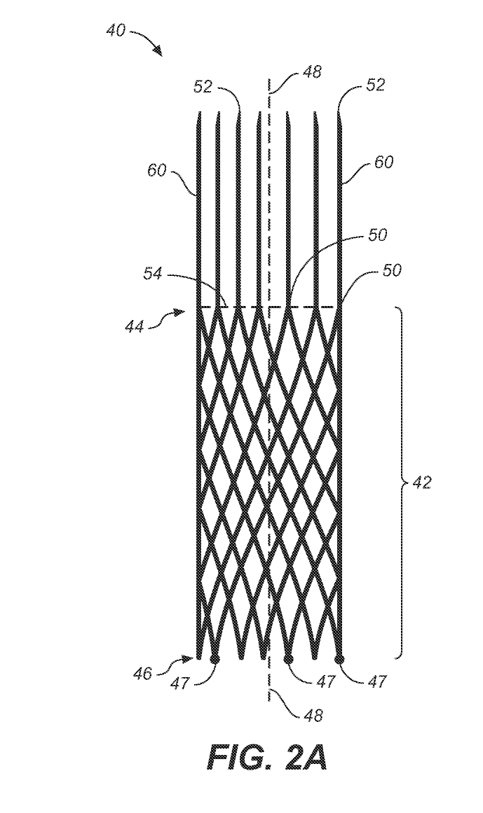

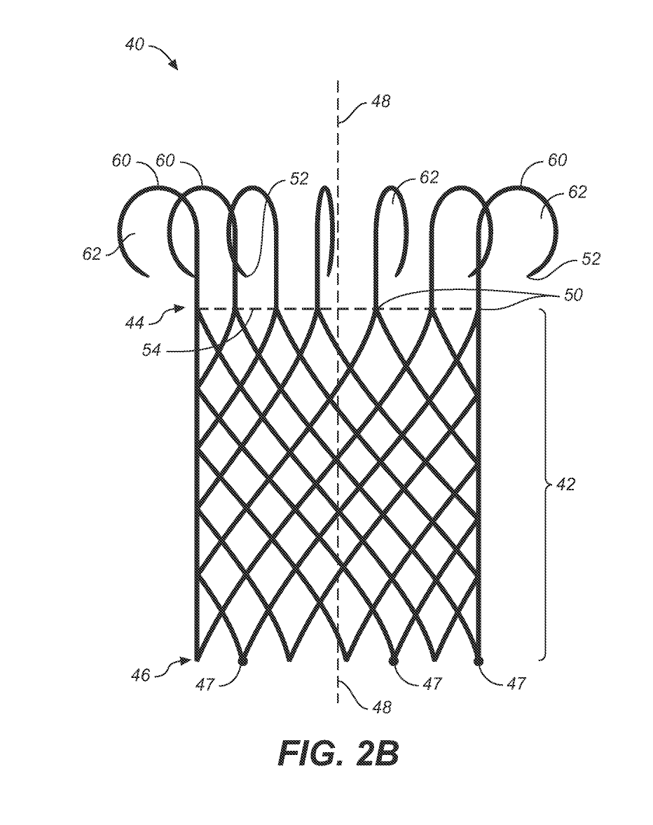

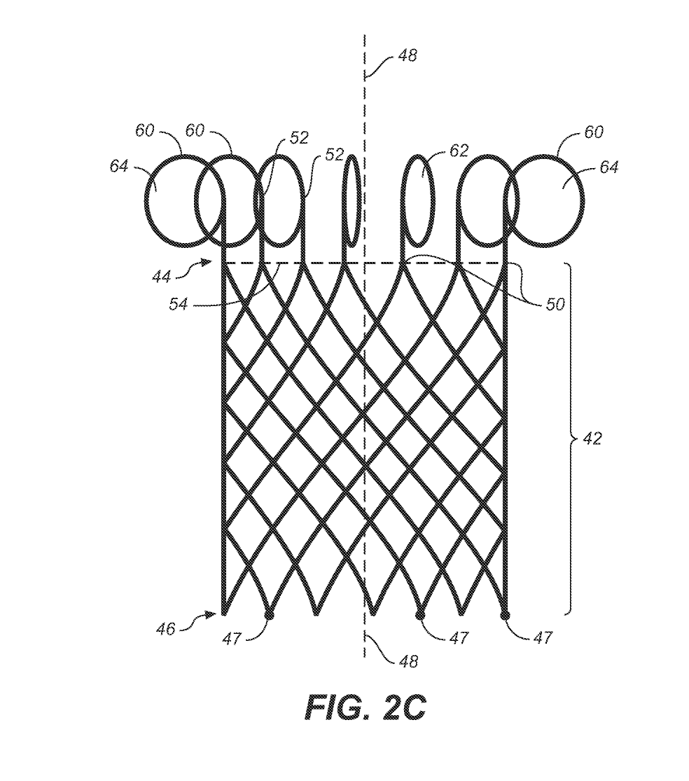

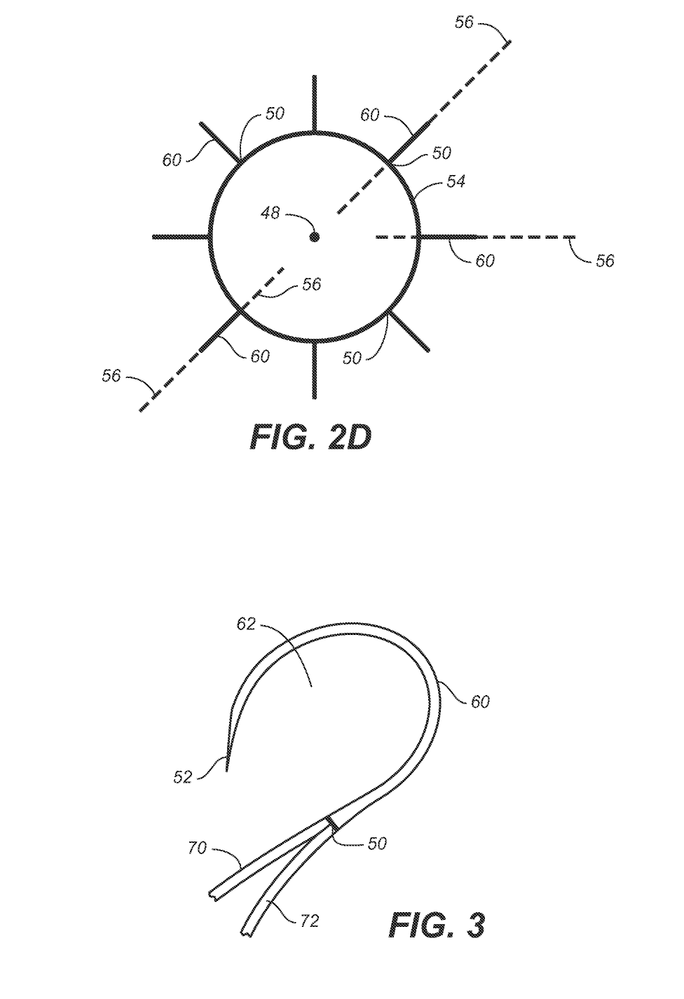

FIGS. 2A-2D, in which like elements share like reference numbers with each other and with FIG. 1, are side and end views of a branch vessel suture stent. FIGS. 2A, 2B, 2C, and 2D illustrate the branch vessel suture stent in the stressed (compressed), two intermediate, and parent (relaxed) states, respectively. FIGS. 2D is an end view of the branch vessel suture stent in the parent state. The branch vessel suture stent 40 includes a stent body 42 and a number of shape memory hooks 60. The branch vessel suture stent 40 is delivered to a branch vessel in the stressed state of FIG. 2A, The hook loops are held by hypotubes 35, FIG. 2B and once released from the hypotubes pass quickly through the intermediate state of FIG. 2C, and relaxes to the parent state of FIGS. 2D when deployed.

FIG. 2A illustrates the stressed state for the branch vessel suture stent 40, in which the diameter of the branch vessel suture stent 40 is compressed to fit within the lumen of a delivery catheter to allow delivery to the branch vessel. The stent body 42 has a first end 44, a second end 46, and a central axis 48. The stent body 42 also has a periphery 54 at the circumference of the first end 44, indicated by the horizontal dashed line. A number of shape memory hooks 60 are connected to the first end 44 at attachment points 50 around the periphery 54. Each of the shape memory hooks 60 has a sharp tip 52. In the stressed state, the shape memory hooks 60 are elongated held within hypotubes (35) so the shape memory hooks 60 are substantially parallel to the central axis 48. As defined herein, elongated indicates a substantially linear configuration for the shape memory hooks 60, which can include one or more curves or bends to place the sharp tips 52 at a desired angle for a particular application. In one embodiment, dissolvable restraining hypotubes can encircle each shape memory hook and a dissolvable restraint band can encircle the whole the branch vessel suture stent 40 to hold the hooks straight and the branch vessel suture stent 40 compressed until the restraining tubes and bands contact fluid in the vasculature. The branch vessel suture stent 40 can proceed to re-configure from the stressed state to the parent state after the restraining bands dissolve and later when the dissolved restraint tube holding each hook straight dissolves.

FIG. 2B illustrates a momentary (instantaneously) intermediate state for the branch vessel suture stent 40, in which the diameter of the branch vessel suture stent 40 has already been expanded and shape memory hooks 60 having just being released from their respective hypotubes and the hook have turned substantially to form partial loops 62. As the stent body 42 of the branch vessel suture stent 40 is disposed in the branch vessel the release of the hooks from the hypotubes causes the partial loops 62 to engage the main vessel stent graft material of a main vessel stent graft and optionally the ostium of the branch vessel.

FIG. 2C illustrates the parent state for the branch vessel suture stent 40, in which the shape memory hooks 60 have formed loops 64.

FIG. 2D is an end view from the end of the branch vessel suture stent 40 to which the shape memory hooks 60 are attached. The parent state is the deployed state for the branch vessel suture stent 40. The shape memory hooks 60 are disposed about the periphery 54 of the first end 44 of the branch vessel suture stent 40 and attached at the attachment points 50. Each of the loops 64 defines a loop plane 56, the projection of which is indicated by dashed lines for some of the shape memory hooks 60 in FIG. 2D. The periphery 54 at the attachment point 50 for each of the shape memory hooks 60 is substantially orthogonal to the loop plane 56 for each of the shape memory hooks 60 in the parent state.

The branch vessel suture stent 40 can be made of any shape memory material having a stressed state and a parent state, such as a shape memory metal and/or shape memory polymer. Examples of shape memory metals include nickel titanium alloys (Nitinol), and the like. The shape memory materials have an Af transformation temperature below body temperature at which the branch vessel suture stent 40 is released from the stressed state and relaxes into the parent state.

The branch vessel suture stent 40 can be formed from a single workpiece or assembled from multiple pieces. In one example, the branch vessel suture stent 40 is laser cut from a single piece of Nitinol tubing, and then the shape memory hooks are formed into loops, treated to set the parent shape, and then elongated into the stressed shape for delivery. In another example, the stent body of the branch vessel suture stent 40 is formed of braided strands of Nitinol woven into a tubular lattice and the shape memory hooks are made of shape set Nitinol tubing sharpened to form the sharp tip and crimped onto one end of the lattice. In another example, the stent body of the branch vessel suture stent 40 is made of one material, such as shape memory polymer, and the shape memory hooks are made of another material, such as shape memory metal. In yet another example, the body stent is balloon expandable and is made of a deformable material, such as steel, stainless steel, cobalt chromium alloys, titanium, polymers, copolymers, or combinations thereof, and the shape memory hooks are made of a shape memory material. Those skilled in the art will appreciate that the pattern of the body stent of the branch vessel suture stent 40 can be any pattern supporting a compressed configuration and an expanded configuration. The pattern can be diamond-shaped, slotted, rectangular, or any other pattern suitable for the desired application.

The hypotubes are made of a stiff, yet thin walled material, such as nitinol or stainless steel, or polymer tubes, which prevent the hooks from bending, but allow the hypotube bundle to expand radially as the proximal sheath 34′ is removed from around it.

The branch vessel suture stent 40 can support suture stent graft fabric material to prevent flow across the stent body and to form a seal with the main vessel stent graft material of the main vessel stent graft deployed in the main vessel. In one embodiment, the suture stent graft fabric material is disposed over and supported by the stent body and the elongated shape memory hooks. The suture stent graft fabric material can include gussets to accommodate motion of the shape memory hooks as required to allow motion of the shape memory hooks between the stressed state and the parent state. If desired for a particular application, the sharp tips of the shape memory hooks can extend through the suture stent graft fabric material when the branch vessel suture stent 40 is in the stressed state. In another embodiment, the suture stent fabric graft material is disposed over the stent body and not disposed over the elongated shape memory hooks. In yet another embodiment, the branch vessel suture stent 40 does not include suture stent graft material. Those skilled in the art will appreciate that the suture stent fabric graft material can be any woven or interlocked graft material suitable for stent grafts, such as woven polymer materials, e.g., Dacron, or polytetrafluoroethylene (PTFE), or interlocked graft materials including knit, stretch, and velour materials.

The branch vessel suture stent 40 can include radiopaque markers as desired to improve visibility on X-ray and fluoroscopic images. The radiopaque markers can be made of platinum, iridium, or any other radiopaque material. In one embodiment, radiopaque marker (disks, or sleeves or coatings) 47 are provided the second end 46 of the stent body 42. Radiopaque marker elements 47 can be provided at other or additional places on the branch vessel suture stent 40, such as at the first end 44.

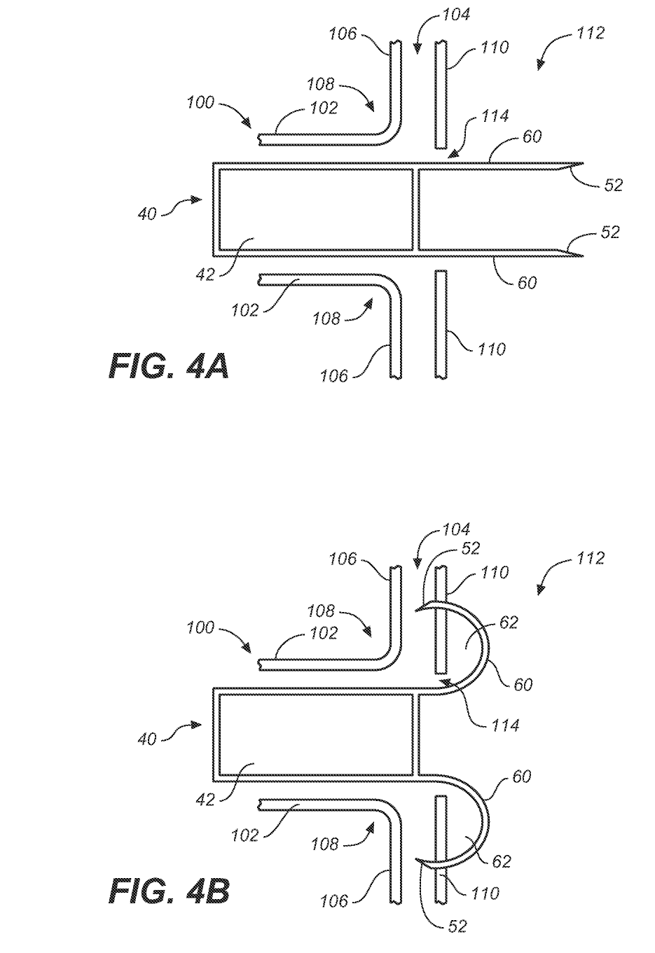

FIG. 3, in which like elements share like reference numbers with FIG. 2A-D, is a detailed view of a shape memory hook for a branch vessel suture stent. The shape of the memory hook as it is released and is moving to its relaxed configuration is illustrated. In this embodiment, the stent body of the branch vessel suture stent is formed of braided strands 70, 72 of shape memory material woven into a cylindrical lattice. The shape memory hook is made of shape memory material cut at a bevel to form the sharp tip 52 and crimped onto the braided strands 70, 72 at the attachment point 50. The diameter of the final loop in the stressed state can be selected for the particular application desired. A small loop diameter, such as 0.15 inches, can be used when the shape memory hooks engage the main vessel stent graft material of the main vessel stent graft, while a larger loop diameter, as large as 0.4 inches, can be used when the shape memory hooks engage both the main vessel stent graft material of the main vessel stent graft and the ostium of the branch vessel.

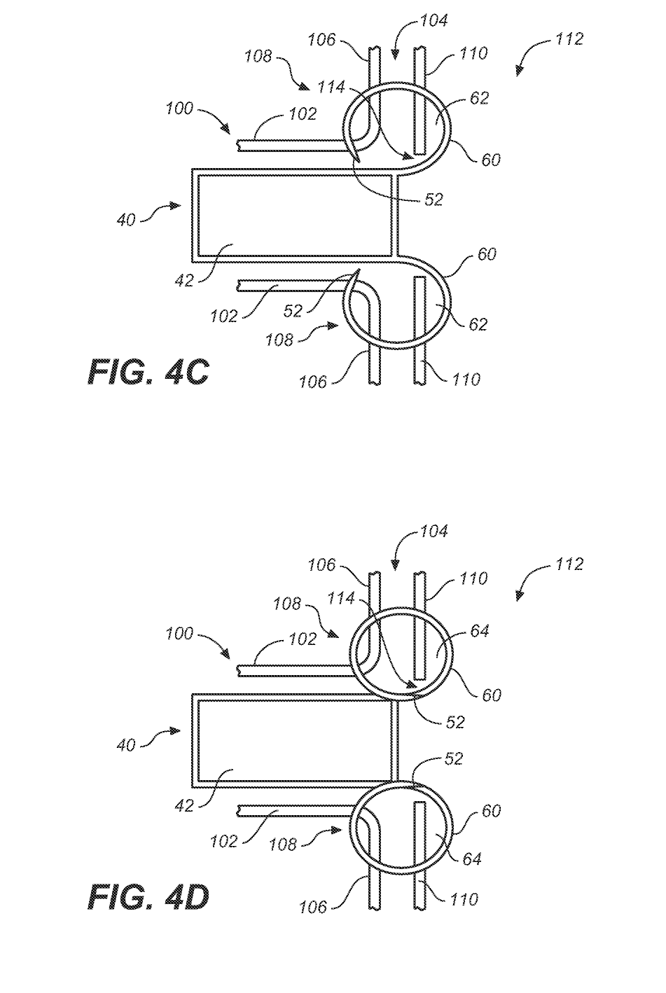

FIGS. 4A-4D, in which like elements share like reference numbers with each other and with FIGS. 2A-2D, are schematic diagrams of vessel/graft engagement of a branch vessel suture stent. In this embodiment, the shape memory hooks of the branch vessel suture stent engage both the main vessel stent graft material of the main vessel stent graft and the ostium of the branch vessel. Only two of the shape memory hooks are shown for clarity of illustration. Those skilled in the art will appreciate that the ostium includes the general area where the branch vessel meets the main vessel and can vary from patient to patient.

Referring to FIG. 4A, the branch vessel 100 having branch vessel walls 102 is connected to the main vessel 104 having main vessel walls 106 at the ostium 108 of the branch vessel 100. A main vessel stent graft including main vessel stent graft material 110 and a main vessel stent graft lumen 112 is deployed in the main vessel 104. A fenestration 114 is formed in the main vessel stent graft material 110. The distal portion of the branch vessel suture stent 40 is positioned through the fenestration 114 with the stent body 42 in the branch vessel 100 and the shape memory hooks 60 in the main vessel stent graft lumen 112. The branch vessel suture stent 40 is in a compressed state with the shape memory hooks 60 held substantially parallel to the central axis of the branch vessel suture stent 40 by hypotubes of the delivery system.

FIGS. 4B-4C, show the configuration of the shape memory hooks at an intermediate snapshot in time. The hooks having just been released from the hypotubes and as a result have begun their movement toward full relaxation (i.e., their original shape set configuration). The shape memory hooks 60 have already partially relaxed to engage the main vessel stent graft material 110 and form a partial loop 62. In FIG. 4C, the shape memory hooks 60 have relaxed further to engage the ostium 108. In FIG. 4D, the shape memory hooks 60 are fully relaxed with the branch vessel suture stent 40 in the parent state to form loops 64. Those skilled in the art will appreciate that FIGS. 4A-4D are simplified to show the interrelation of the parts. For example, the stent body 42 actually contacts the branch vessel walls 102 to maintain patency of the branch vessel 100. The loops 64 fix the main vessel stent graft to the branch vessel so that the fenestration is aligned with the branch vessel, and seal the main vessel stent graft material 110 against the ostium 108.

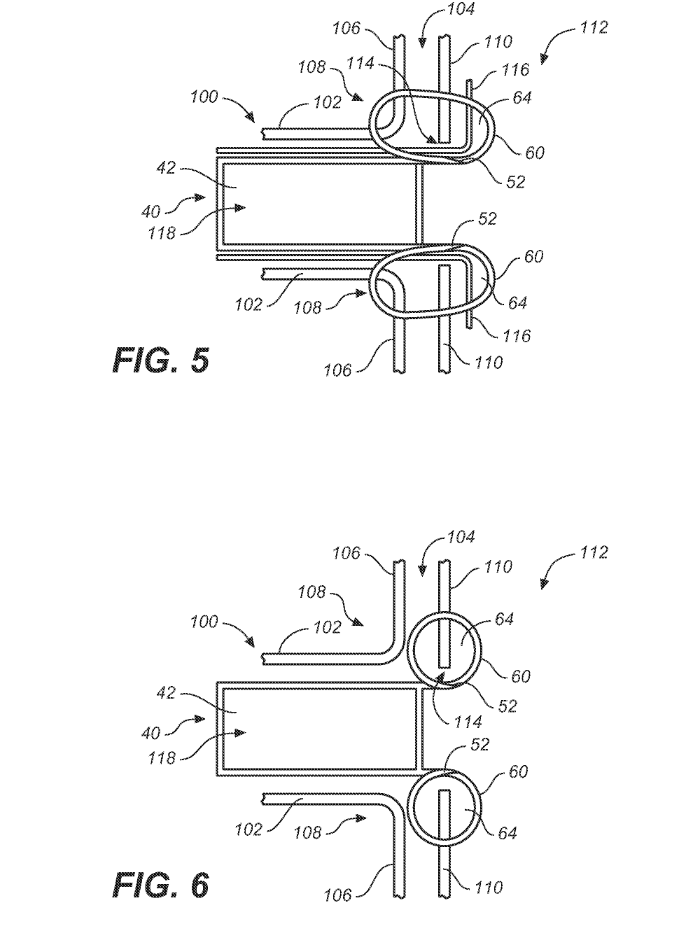

FIG. 5, in which like elements share like reference numbers with FIG. 4D, is a schematic diagram of vessel/graft engagement including suture stent graft material of a branch vessel suture stent made in accordance with the present invention. In this example, the branch vessel suture stent 40 includes suture stent graft material 116 illustrated in cross section. In the stressed state of the branch vessel suture stent 40, the suture stent graft material 116 surrounds the elongated shape memory hooks 60, and the sharp tips 52 of the shape memory hooks 60 pass through the suture stent graft material 116. In the parent state of the branch vessel suture stent 40, the loops 64 engage the suture stent graft material 116, the main vessel stent graft material 110, and the ostium 108. The loops 64 fix the main vessel stent graft to the branch vessel 100 so that the fenestration 114 is aligned with the branch vessel 100. The loops 64 also seal the main vessel stent graft material 110 against the ostium 108 and seal the main vessel stent graft material 110 against the suture stent graft material 116.

FIG. 6, in which like elements share like reference numbers with FIG. 4D, is a schematic diagram of graft engagement of a branch vessel suture stent. In this example, the loops 64 engage the main vessel stent graft material 110, but not the ostium 108. The diameter of the loop 64 can be smaller for the case when the loop only engages the main vessel stent graft material, compared to the loop diameter for a loop engaging the main vessel stent graft material and the ostium. The loops 64 fix the main vessel stent graft to the branch vessel 100 so that the fenestration 114 is aligned with the branch vessel 100. When the branch vessel suture stent 40 has suture stent graft material (not shown) disposed about the shape memory hooks 60 and the stent body 42, the main vessel stent graft material 110 and the suture stent graft material provide a sealed flow path between the main vessel stent graft lumen 112 and the suture stent lumen 118.

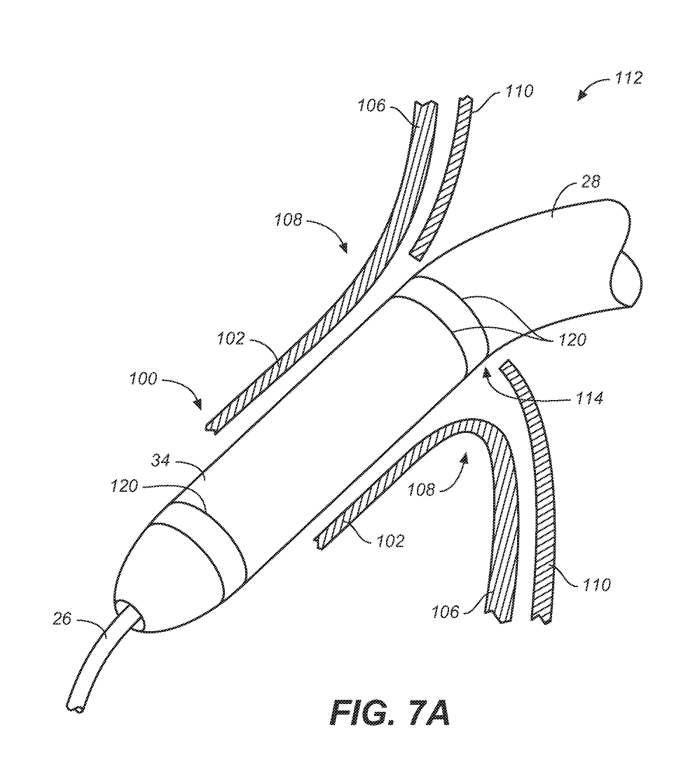

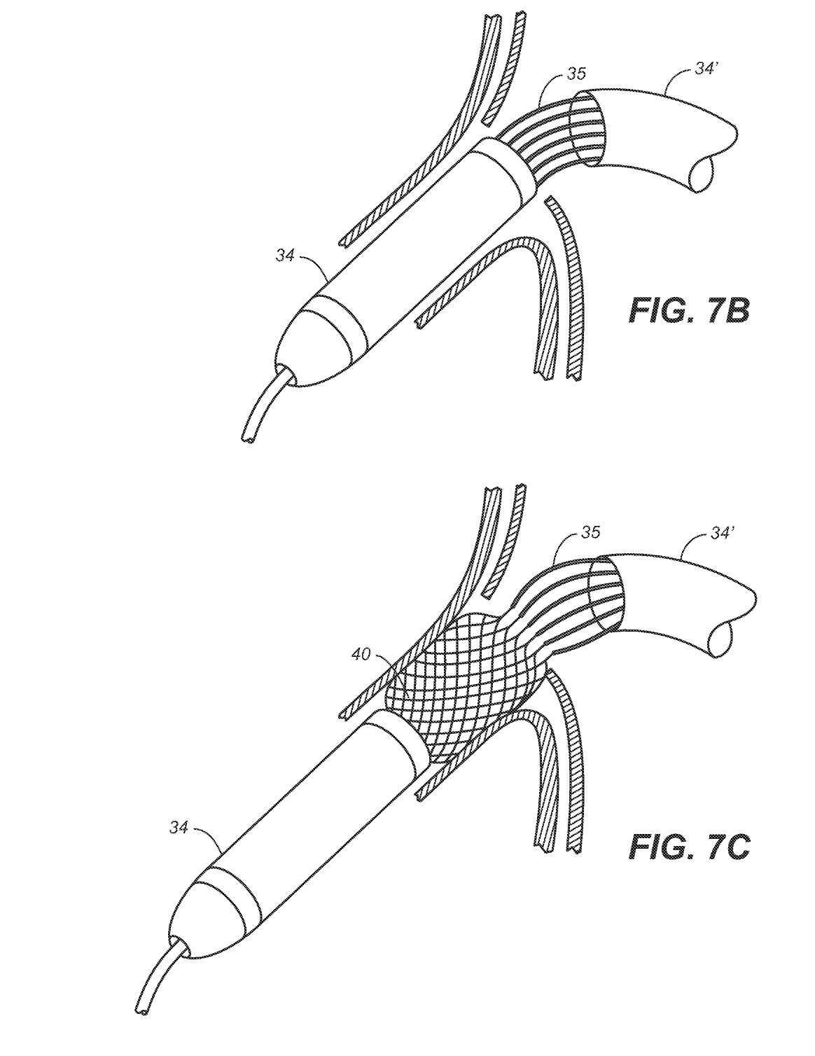

FIGS. 7A-C, in which like elements share like reference numbers with FIGS. 1A-6, are schematic views of deployment of a branch vessel suture stent. In this example, the hypotubes 35 are emerging as the proximal sheath 34′ is retracted and as the distal sheath 34 is pushed distally into the branch vessel 100. The hypotubes expand with the main body of the stent to allow the hooks 64 when released to engage the main vessel stent graft material 110. In this embodiment, radiopaque marker bands 120 (at the distal end of the proximal sheath 34′, the proximal end of the distal sheath 34, and near the tip at the distal end of the distal sheath 34) are located on the sheath (34, 34′) to improve visibility on X-ray and fluoroscopic images.

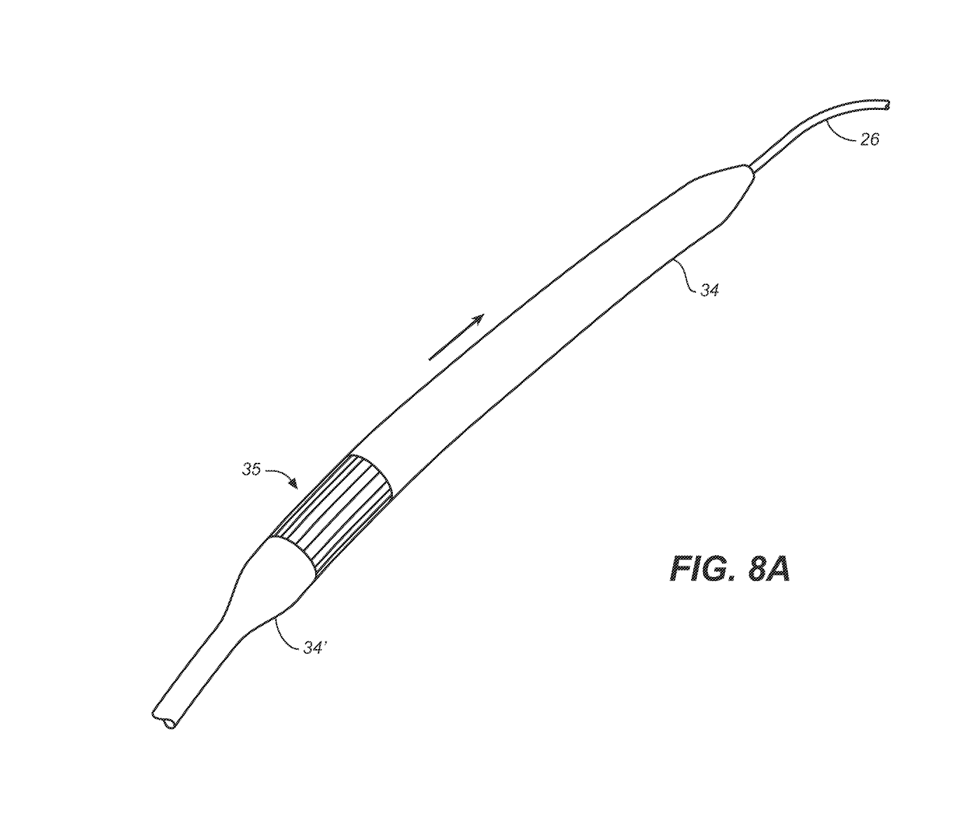

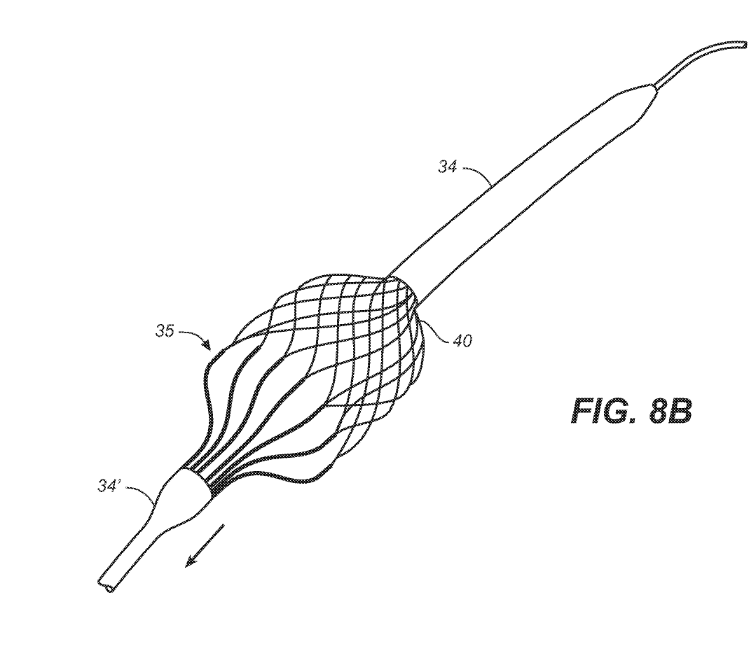

FIGS. 8A and B are alternate schematicized perspective views showing the retraction of the sheath from the hypotubes and the hypotube bundle expansion along with the main body portion of the stent, prior to retraction and release of the hypotubes, which then release the hooks, which are then disposed at their radial diameter adjacent the ostium opening and in proximity to the branch vessel wall. Without this hypotube radial expansion the hooks could not be released in a position where they would have an opportunity to pierce the perimeter of the fenestration in the branch vessel and the underlying branch elements and vessel walls.

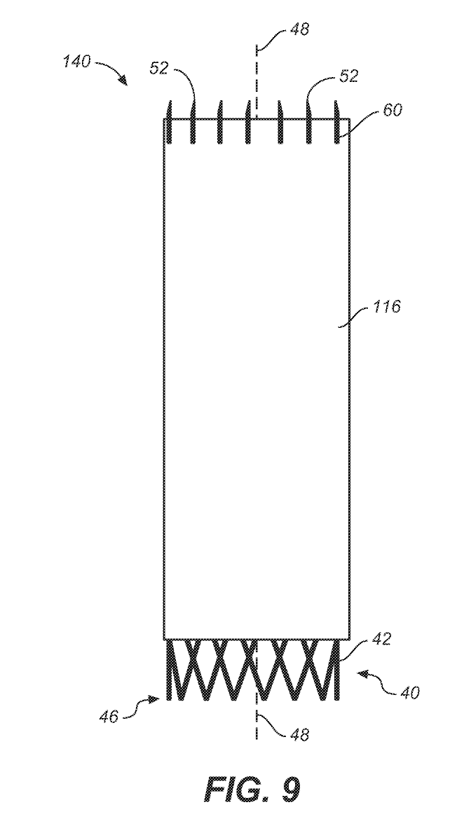

FIG. 9, in which like elements share like reference numbers with FIGS. 2A-D and 5, is a side view of a branch vessel suture stent with suture stent graft material. In this example, the covered branch vessel suture stent 140 is shown in the stressed state. Suture stent graft material 116 is disposed about the branch vessel suture stent 40. The sharp tips 52 of the shape memory hooks 60 pass through the suture stent graft material 116. The suture stent graft material 116 can cover the stent body 42 partially or completely. The suture stent graft material 116 can be attached to the branch vessel suture stent 40 with adhesive, stitching, or the like, inside or outside of the branch vessel suture stent 40 as desired for a particular application.

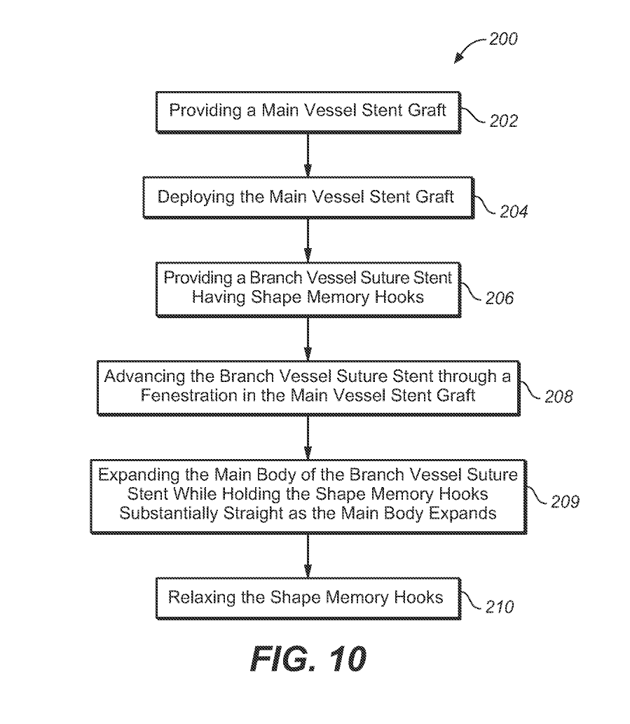

FIG. 10 is a flowchart of a method of stenting a branch vessel with a branch vessel suture stent made in accordance with the present invention. The method 200 includes providing a main vessel stent graft 202, deploying the main vessel stent graft 204, providing a branch vessel suture stent having shape memory hooks 206, advancing the branch vessel suture stent through a fenestration in the main vessel stent graft 208, expanding the main body of the branch vessel suture stent while holding the shape memory hooks substantially straight as the main body expands 209, and relaxing the shape memory hooks 210.

The providing a main vessel stent graft 202 includes providing a main vessel stent graft having main vessel stent graft material and a main vessel stent graft lumen. In one embodiment, the main vessel stent graft also has a fenestration pre-formed in the main vessel stent graft material at the anticipated axial location of the branch vessel. The fenestration can be located based on measurements of the anatomy of the particular patient.

The deploying the main vessel stent graft 204 includes deploying the main vessel stent graft in the main vessel. In one embodiment, the main vessel stent graft is advanced to the deployment site in the main vessel through the femoral artery, the carotid artery, or the subclavian artery. In one embodiment, the deploying the main vessel stent graft 204 further includes fenestrating the main vessel stent graft to form the fenestration at the location of the ostium of the branch vessel.

The providing a branch vessel suture stent having shape memory hooks 206 includes providing a branch vessel suture stent in a stressed state, the branch vessel suture stent having a stent body and shape memory hooks, the stent body having a first end including a first periphery, and the shape memory hooks being disposed about the first periphery. An exemplary branch vessel suture stent is illustrated in FIG. 2A-D. The branch vessel suture stent can also include suture stent graft material.

Referring to FIG. 10, the advancing the branch vessel suture stent through a fenestration in the main vessel stent graft 208 includes advancing the branch vessel suture stent through a fenestration in the main vessel stent graft to locate the stent body in the branch vessel and the shape memory hooks in the main vessel stent graft lumen.

The expanding the main body of the branch vessel suture stent while holding the shape memory hooks substantially straight as the main body expands 209 involves the radial expansion of the hypotube buddle (array) to position the hooks adjacent to their points of engagement with the adjacent structures prior to deployment. The relaxing the shape memory hooks 210 includes relaxing each of the shape memory hooks to engage the main vessel stent graft material and form a loop. The shape memory hooks 210 relax from a stressed state to a parent state when the shape memory is above the transformation temperature. The shape memory hooks 210 are restrained by hypotubes and/or dissolvable restraining bands until the branch vessel suture stent is in place for deployment.

While specific embodiments are disclosed herein, various changes and modifications can be made without departing from the spirit and scope thereof.

1. A branch vessel suture stent comprising:
a stent body having a first end, a second end, and a central axis, the first end having a first periphery; and
shape memory hooks disposed about the first periphery, each of the shape memory hooks being attached to the first periphery at an attachment point, the shape memory hooks being elongated in a stressed state and looped in a parent state, each of the shape memory hooks defining a loop plane in the parent state;
wherein the shape memory hooks are substantially parallel to the central axis in the stressed state, and the first periphery at the attachment point for each of the shape memory hooks is substantially orthogonal to the loop plane for each of the shape memory hooks in the parent state.

2. The branch vessel suture stent of claim 1 wherein the stent body comprises braided strands woven into a tubular lattice.

3. The branch vessel suture stent of claim 2 wherein the shape memory hooks comprise shape memory material tubing having a first end and a second end, the shape memory material tubing being sharpened on the first end to form a sharp tip and crimped onto the braided strands on the second end.

4. The branch vessel suture stent of claim 1 wherein the stent body and the shape memory hooks are formed of a single piece of laser cut shape metal tubing.

5. The branch vessel suture stent of claim 1 wherein the stent body is made of a shape memory material selected from the group consisting of nickel titanium alloy and shape memory polymers.

6. The branch vessel suture stent of claim 1 wherein the stent body is made of a deformable material selected from the group consisting of steel, stainless steel, cobalt chromium, titanium, polymers, copolymers, and combinations thereof.

7. The branch vessel suture stent of claim 1 wherein the shape memory hooks are made of a shape memory material selected from the group consisting of nickel titanium alloy and shape memory polymers.

8. The branch vessel suture stent of claim 1 wherein the stent body is made of one material and the shape memory hooks are made of another material.

9. The branch vessel suture stent of claim 1 further comprising a suture stent graft material supported by the stent body and the shape memory hooks.

10. The branch vessel suture stent of claim 9 wherein the shape memory hooks pass through the suture stent graft material.

11. The branch vessel suture stent of claim 9 wherein the suture stent graft material is made of a material selected from the group consisting of woven graft materials, woven polymer materials, and interlocked graft materials.

12. The branch vessel suture stent of claim 1 further comprising a radiopaque marker disposed on the stent body.

13. A branch vessel suture stent system comprising:
a suture stent delivery catheter; and
a branch vessel suture stent operably attached to the suture stent delivery catheter, the branch vessel suture stent having a stent body having a first end, a second end, and a central axis, the first end having a first periphery; and shape memory hooks disposed about the first periphery, each of the shape memory hooks being attached to the first periphery at an attachment point, the shape memory hooks being elongated in a stressed state and looped in a parent state, each of the shape memory hooks defining a loop plane in the parent state;
wherein the shape memory hooks are held substantially parallel to the central axis by separate hypotubes in the stressed state, and the first periphery at the attachment point for each of the shape memory hooks is substantially orthogonal to the loop plane for each of the shape memory hooks in the parent state.

14. The branch vessel suture stent system of claim 13 wherein the stent body comprises braided strands woven into a tubular lattice.

15. The branch vessel suture stent system of claim 14 wherein the shape memory hooks comprise shape memory material tubing having a first end and a second end, the shape memory material tubing being sharpened on the first end to form a sharp tip and crimped onto the braided strands on the second end.

16. The branch vessel suture stent system of claim 13 wherein the stent body and the shape memory hooks are formed of a single piece of laser cut shape metal tubing.

17. The branch vessel suture stent system of claim 13 wherein the stent body is made of a shape memory material selected from the group consisting of nickel titanium alloy and shape memory polymers.

18. The branch vessel suture stent system of claim 13 wherein the stent body is made of a deformable material selected from the group consisting of steel, stainless steel, cobalt chromium, titanium, polymers, copolymers, and combinations thereof.

19. The branch vessel suture stent system of claim 13 wherein the shape memory hooks are made of a shape memory material selected from the group consisting of nickel titanium alloy and shape memory polymers.

20. The branch vessel suture stent system of claim 13 wherein the stent body is made of one material and the shape memory hooks are made of another material.

21. The branch vessel suture stent system of claim 13 further comprising a suture stent graft material supported by the stent body and the shape memory hooks.

22. The branch vessel suture stent system of claim 21 wherein the shape memory hooks pass through the suture stent graft material.

23. The branch vessel suture stent system of claim 21 wherein the suture stent graft material is made of a material selected from the group consisting of woven graft materials, woven polymer materials, and interlocked graft materials.

24. The branch vessel suture stent system of claim 13 further comprising a radiopaque marker disposed on the stent body.

25. The branch vessel suture stent system of claim 13 wherein the suture stent delivery catheter has a retractable sheath and the branch vessel suture stent is operably attached to the suture stent delivery catheter by the retractable sheath.

26. The branch vessel suture stent system of claim 25 further comprising a radiopaque marker disposed on the retractable sheath.

27. A method of stenting a branch vessel off a main vessel, the method comprising:
providing a main vessel stent graft having main vessel stent graft material and a main vessel stent graft lumen;
deploying the main vessel stent graft in the main vessel;
providing a branch vessel suture stent in a stressed state, the branch vessel suture stent having a stent body and shape memory hooks, the stent body having a first end including a first periphery, the shape memory hooks being disposed about the first periphery;
advancing the branch vessel suture stent through a fenestration in the main vessel stent graft to locate the stent body in the branch vessel and the shape memory hooks in the main vessel stent graft lumen;
expanding the main body while holding the hooks stressed, and
relaxing each of the shape memory hooks to engage the main vessel stent graft material and form a loop.

28. The method of claim 27 wherein the branch vessel has an ostium and the relaxing further comprises relaxing each of the shape memory hooks through the ostium.

29. The method of claim 27 wherein the providing a main vessel stent graft further comprises providing a main vessel stent graft having the fenestration.

30. The method of claim 27 wherein the deploying the main vessel stent graft further comprises fenestrating the main vessel stent graft to form the fenestration.

31. The method of claim 27 wherein the advancing comprises advancing the branch vessel suture stent from the main vessel to the branch vessel.

32. The method of claim 27 wherein the advancing comprises advancing the branch vessel suture stent from the branch vessel to the main vessel.

33. A branch vessel suture stent for use in a branch vessel with a main vessel stent graft having main vessel stent graft material, the branch vessel suture stent comprising:
means for stenting the branch vessel, the stenting means having a central axis; and
means for hooking the main vessel stent graft material, the hooking means being connected to the stenting means and having a stressed state and a parent state;
wherein each of the hooking means is substantially parallel to the central axis in the stressed state and forms a loop in the parent state.

34. The branch vessel suture stent of claim 33 wherein the branch vessel has an ostium and the hooking means further comprises means for hooking the ostium.

35. The branch vessel suture stent of claim 34 wherein the main vessel stent graft is deployed in a main vessel and the means for hooking the ostium further comprises means for fixing the main vessel stent graft in the main vessel relative to the branch vessel.

36. The branch vessel suture stent of claim 33 further comprising suture stent graft material disposed about the stenting means and the hooking means.

37. The branch vessel suture stent of claim 36 wherein the hooking means further comprises means for sealing the main vessel stent graft material and the suture stent graft material.
